(19) United States
(12) Patent Application Publication
(10) Pub. No.: US 2014/0050774 A1
(43) Pub. Date: Feb. 20, 2014

(54) WOUND TREATMENT THERAPY

(71) Applicant: INTEGRATED HEALING TECHNOLOGIES, LLC, FRANKLIN, TN (US)

(72) Inventors: ALBERT RODEWALD, FRANKLIN, TN (US); ROBERT STEPHEN PORTER, BRENTWOOD, TN (US)

(73) Assignee: INTEGRATED HEALING TECHNOLOGIES, LLC, FRANKLIN, TN (US)

(21) Appl. No.: 14/061,672

(22) Filed: Oct. 23, 2013

Related U.S. Application Data
(63) Continuation of application No. 13/534,597, filed on Jun. 27, 2012, now abandoned.
(60) Provisional application No. 61/571,366, filed on Jun. 27, 2011.

(51) Int. Cl. A61K 33/20; A61K 9/00

(52) U.S. Cl. CPC A61K 33/20; A61K 9/0014
USPC 424/445; 424/661

(57) ABSTRACT

The wound treatment therapy deactivates pathogenic microorganisms within the wound environment without disrupting the cell walls. The wound treatment therapy of this invention combines the application of stabilized hypochlorous acid compound with the application of a hydrophobic wound dressing. The application of hypochlorous acid provides the initial anti-microbial agent, which disrupts and eradicates biofilm, as well as, deactivates the harmful pathogenic organisms within the wound environment. The application of the hydrophobic wound dressing, which is designed to bind harmful hydrophobic microorganisms and toxins, provides a mechanical vehicle for physically removing deactivated hydrophobic microorganisms form the wound environment with each dressing change.

This application claims the benefit of U.S. Provisional Patent Application, Ser. No. 61/571,366 filed Jun. 27, 2011, the entirety of which is incorporated by reference herein.

### BACKGROUND OF THE INVENTION

This invention relates to a wound treatment therapy, and in particular a wound treatment methodology that deactivates pathogenic microorganisms without disrupting cell walls or building antibiotic resistance.

In wound care treatment, it is desirable to both deactivate pathogenic microorganisms within the wound environment and physically remove those deactivated microorganisms, as well as, other harmful toxins, from the wound environment. Antibiotics and other drugs are useful in destroying bacteria and other pathogens, but their operation disrupts the cell walls of the microorganisms, releasing harmful enzymes and other toxins into the wound environment. Without a mechanism for their physical removal from the wound environment, such harmful enzymes and toxins can result in cytotoxicity.

It is also known that when planktonic, free floating, individual microorganisms, like bacteria, attach to a surface and gather in groups within a wound environment and else where, they produce an extra-cellular matrix called a biofilm. Biofilms are held together and protected by a matrix of excreted polymeric compounds called EPS (exopolysacchride). Biofilms provide a protective environment for microorganisms that exist within the biofilm mass and a level of immunity from antibiotic treatments, except in doses that would be fatal to the patient. Consequently, biofilm microorganisms are not affected by the body's own infection-resistance mechanisms. In addition, bacterial biofilms may impair cutaneous wound healing and reduce topical antibacterial efficiency in healing or treating infected skin wounds.

Hypochlorous acid (HOCl) is the active component found in our own immune system's natural defense against infection, and thus kills bacteria, fungi and viruses without creating mechanisms for drug resistance. Hypochlorous acid compounds can be a safe, fast acting, broad spectrum antimicrobial, for topical application, effective against all groups of hydrophobic, hydrophilic, gram-negative and gram-positive bacteria, and yeast, including pseudomonas aeruginosa, Staphylococcus aureus, and multi-drug resistant organisms like methicillin resistant staphylococcus aureus (MRSA), and vancomycin resistant enterococci (VRE). In addition, hypochlorous acid compounds rapidly penetrate and disrupt biofilm and eradicate the pathogens, the major factor that inhibits wound healing. Unlike most antibiotics, which can take many hours to work, hypochlorous acid compounds, even at small doses, can help eradicate bacteria, fungi and viruses in minutes. When applied topically within a wound environment, hypochlorous acid compounds immediately disrupt and eradicate the bio-film, which surrounds and deactivates a broad spectrum of pathogenic microorganisms. Hypochlorous acid generally controls the tissue bacterial bio-burden without inhibiting the wound healing process and is also commonly associated with improved wound closure.

Hydrophobic wound dressings, such as the ones described in U.S. Pat. No. 4,617,326 and available from Abigo Medical AB under the Sorbact® brand dressings, have proven effective against hydrophobic microorganisms. Hydrophobic wound dressings, such as Sorbact, generally consist of a cellulose acetate fabric treated with an application of dioctadecyl carbamoyl chloride DACC of AKD, which creates a covalent bond between the materials. The mechanism of action for such hydrophobic wound dressings is based upon the physical effect of hydrophobic interaction, whereby water repellant substances bind together in an aqueous environment. Since most pathogenic microorganisms are hydrophobic, they bind to the dressing and are inactivated and removed from the wound with the dressing change. In addition, hydrophobic wound dressing can prevent the formation of biofilm, but do not destroy existing biofilm matrix.

### SUMMARY OF THE INVENTION

The wound treatment therapy of this invention deactivates pathogenic microorganisms within the wound environment without disrupting the cell walls, which can release harmful enzymes and other toxins into the wound environment. The wound treatment therapy of this invention combines the application of stabilized hypochlorous acid compound with the application of a hydrophobic wound dressing. The application of hypochlorous acid provides the initial anti-microbial agents, which disrupt and eradicate biofilm, as well as, deactivating harmful pathogenic organisms within the wound environment. The application of the hydrophobic wound dressing, which is designed to bind harmful hydrophobic microorganisms and toxins, provides a mechanical vehicle for physically removing neutralized hydrophobic microorganisms form the wound environment with each dressing change. By combining the application of a hypochlorous acid compound with the application of a hydrophobic wound dressing to a wound environment, the natural antimicrobial actions and effects of both treatments are enhanced.

The above described features and advantages, as well as others, will become more readily apparent to those of ordinary skill in the art by reference to the following detailed description and accompanying drawings.

### BRIEF DESCRIPTION OF THE DRAWINGS

The present invention may take form in various system and method components and arrangement of system and method components. The drawings are only for purposes of illustrating exemplary embodiments and are not to be construed as limiting the invention. The drawings illustrate the present invention, in which:

FIG. 1 is a partial side sectional view of a wound containing hydrophobic pathogenic microorganisms and hydrophilic pathogenic microorganisms and a hydrophobic wound dressing and a representation of an application of hypochlorous acid.

FIG. 2 is a partial side sectional view of the wound of FIG. 1 showing the application of hypochlorous acid into the wound cavity 1 for neutralizing both hydrophobic pathogenic microorganisms and hydrophilic pathogenic microorganisms.

FIGS. 3 and 4 are a partial side sectional view of the wound of FIG. 1 showing the application of a hydrophobic wound dressing into the wound cavity to absorb and bind neutralized hydrophobic pathogenic microorganisms and hydrophilic pathogenic microorganisms.

FIG. 5 is a partial side sectional view of the wound of FIG. 1 illustrating the physical removal of a hydrophobic wound dressing with hydrophobic pathogenic microorganisms bound to the dressing.

FIG. 6 is a partial side sectional view of the wound of FIG. 1 showing the application of a hydrophobic wound dressing into the wound cavity.

FIG. 7 is a partial side view of the wound of FIG. 1 showing a hypochlorous acid compound saturating the hydrophobic wound dressing of FIG. 6.

FIG. 8 is a partial side sectional view of the wound of FIG. 1 illustrating the physical removal of a hydrophobic wound dressing of FIG. 7 with hydrophobic pathogenic microorganisms bound to the dressing.

### DESCRIPTION OF THE PREFERRED EMBODIMENT

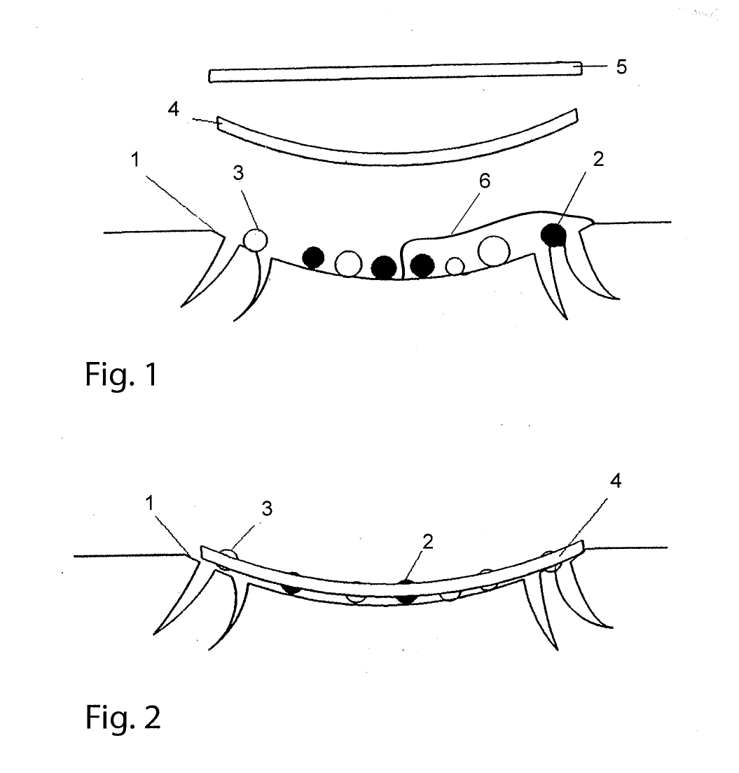

Referring now to the drawings, FIGS. 1-5 illustrate an example of the wound treatment therapy of this invention. FIG. 1 illustrates a wound environment 1 containing hydrophobic pathogenic microorganisms 3 and hydrophilic pathogenic microorganisms 2, which are enclosed and protected by biofilm 6.

FIG. 2 illustrates the application of hypochlorous acid 4 directly into the wound environment 1, which disrupts and eradicates biofilm 6 and deactivates both hydrophobic pathogenic microorganisms 3 and hydrophilic pathogenic microorganisms 2. The particular formulary and composition of hypochlorous Acid compound 4 is selected based on therapeutic affects desired. The hypochlorous acid compound may contain various stabilizers and other compounds to achieve the desired therapeutic properties and may be applied to wound 1, either as a topical solution or gel, as necessary in any particular application. Generally, the volume of hypochlorous compound 4 used in any application within the wound is sufficient to thoroughly irrigate the wound environment.

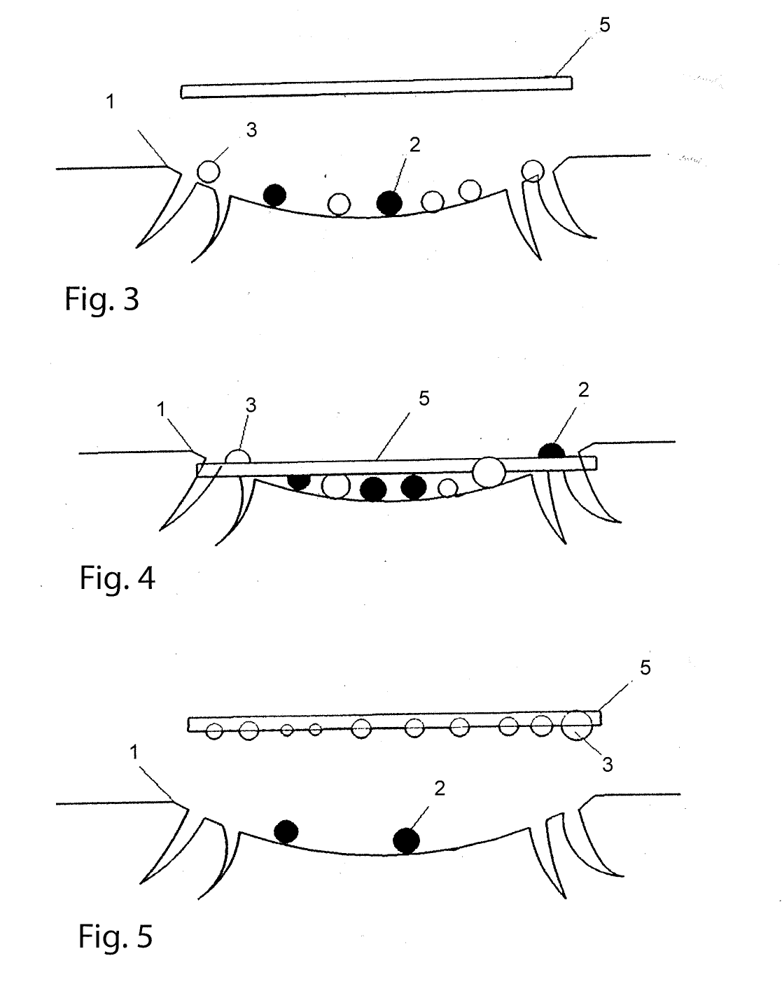

FIG. 3 illustrates wound 1 after the application of hypochlorous acid compound 4 breaks up biofilm 6 leaving deactivated hydrophobic pathogenic microorganisms 3 and hydrophilic pathogenic microorganisms 2 prior to the placement of a hydrophobic wound dressing 5. It should be noted that the bio-chemical operation of hypochlorous acid compound 4 breaks down into chlorine gas that evaporates from the wound environment and water, which irrigates the wound environment.

FIG. 4 shows the application of hydrophobic wound dressing 5 into the wound environment 1. Ideally, hydrophobic wound dressing 5 employs the type of wound dressings and materials described in U.S. Pat. No. 4,617,326, which is incorporated herein by reference, and available from Abigo Medical AB under the Sorbact® brand. Hydrophobic wound dressings 5 generally consist of a cellulose acetate fabric treated with an application of dioctadecyl carbamoyl chloride DACC of AKD, which creates a covalent bond between the materials. Hydrophobic wound dressing 5 may take a variety of forms as necessary for any particular application, and may take the form of a gause, pad, cellular foam, or any combination thereof. For example, hydrophobic wound dressing 5 consist of a cellulose acetate fabric gauze treated with dioctadecyl carbamoyl chloride, which can be packed into wound environment 1. In an other example, hydrophobic wound dressing 5 may consist of a hydrophobic fabric (not shown) encasing a gauze pad of hydrophilic fibers treated with a hydrophobic coating (not shown), whereby the hydrophobic microbes pass through hydrophobic fabric, but are captured within the coated but hydrophilic fibers. In all cases, hydrophobic wound dressing 5 uses hydrophobic interaction to bind water repellant substances and microbes together in an aqueous environment of the dressing. The moisture from the hypochlorous acid compound and the water produced from its therapeutic operation ensures the activation of hydrophobic wound dressing 5.

FIG. 5 shows the physical removal of hydrophobic pathogenic microorganisms 3 upon removal of the hydrophobic wound dressing 5 designed to bind hydrophobic microorganisms 3. Hydrophilic microorganisms 2 remain in the wound. Removing hydrophobic wound dressing 5 physically removes toxins and neutralized microbes from the wound environment.

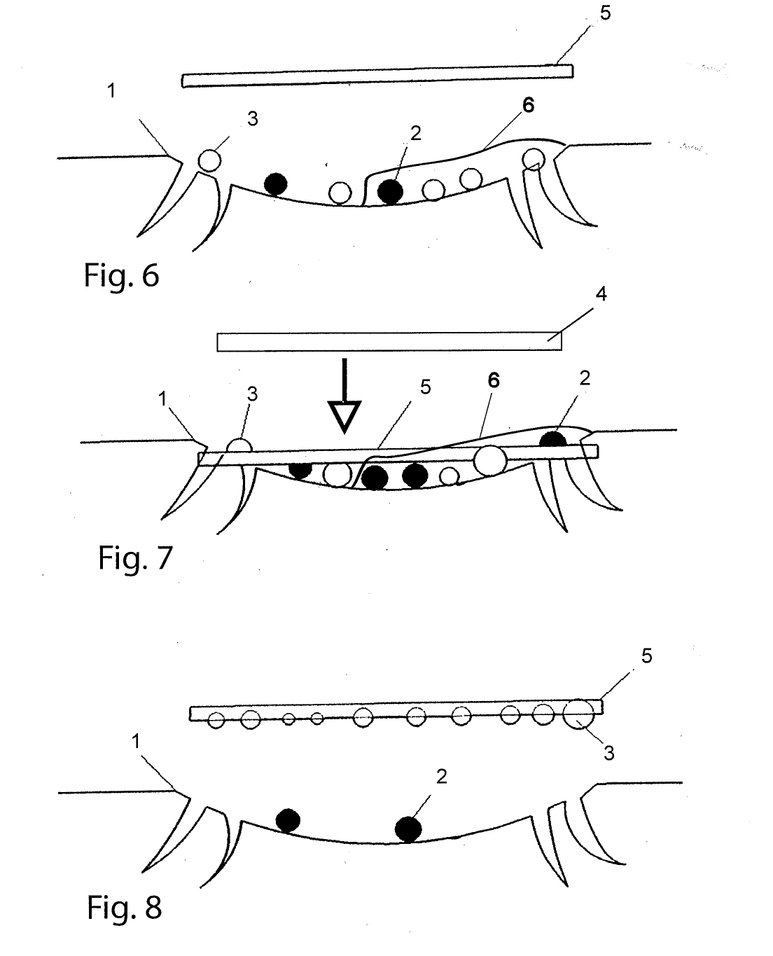

FIGS. 6-8 illustrate an alternative example of the wound treatment therapy of this invention. As shown, the hydrophobic wound dressing 5 is applied to the wound environment 1 first, then saturated with the hypochlorous acid compound 4. The dressing is generally saturated with hypochlorous acid compound 4 to penetrate the dressing and thoroughly irrigate the wound environment 1.

As shown in both examples, the wound treatment therapy consists of topically applying hypochlorous acid to the wound environment and applying a hydrophobic wound dressing to facilitate for continued and sustained cell growth. The topical application of hypochlorous acid immediately disrupts and eradicates biofilm and deactivates pathogenic microorganisms within the wound environment. The application of the hydrophobic wound dressing binds the deactivated hydrophobic microorganisms and other toxins within the dressing itself, so that upon removal of the hydrophobic wound dressing microorganisms and toxins are removed. The combination of the application of hypochlorous acid compounds and hydrophobic wound dressings enhances and prolongs the other's therapeutic properties. In addition, the wound treatment therapy of this invention provides anti-microbial treatment without disrupting the cell walls of the microbes, which eliminates the harmful effects of a lysing pathogens within the wound environment.

The wound treatment therapy of this invention may include in certain applications the use of negative pressure wound therapy, hyperbaric pressure wound therapy, and/or the deliberate introduction of oxygen into the wound environment. The use of negative pressure wound therapy, positive pressure (hyperbaric) pressure therapy and the use of topical oxygen for wound treatment is disclosed in U.S. Pat. No. 7,648,488 filed Nov. 21, 2006, and incorporated by reference in its entirety, herein. The use of negative pressure wound therapy, positive pressure (hyperbaric) wound therapy and the application of topical oxygen to the wound environment all increase the effectiveness of the hypochlorous acid within the wound environment. Negative pressure wound treatment therapy occludes micro vessels in a sealed environment thus depriving the wound of oxygen. In addition, negative pressure wound therapy mechanically removes fluids and microbes from the wound site. Consequently, negative pressure therapy and the application of topical oxygen can be used to manipulate the duration of the effectiveness of the hypochlorous acid. Topical application of oxygen to a wound environment and the use of hyperbaric oxygen chambers increase blood flow to wound environment. In addition, the topical application of oxygen within a wound environment increases cell surface hydrophobicity in anaerobic bacteria. When anaerobic bacteria are subjected to oxygen rich environments, cellular stress is caused, resulting in an increase of cell surface hydrophobicity (CSH). When subjected to an oxygen rich environment, anaerobic microbes already expressing CSH will express higher levels of CSH and anaerobic microbes not expressing CSH will become stressed and begin to exhibit CSH. By deliberately introducing oxygen to a wound environment, the binding effect of the hydrophobic wound dressing is enhanced.

The embodiment of the present invention herein described and illustrated is not intended to be exhaustive or to limit the invention to the precise form disclosed. It is presented to explain the invention so that others skilled in the art might utilize its teachings. The embodiment of the present invention may be modified within the scope of the following claims.

We claim:

1. A wound treatment therapy comprising: a) applying topically a hypochlorous acid compound within a wound environment to deactivate micoorganisms without lysing pathogens; b) applying a hydrophobic wound dressing to the wound environment to bind deactivated microorganisms thereto; and c) removing the hydrophobic wound dressing from the wound environment to remove the deactivated microorganisms.

2. The therapy of claim 1 wherein the hypochlorous acid is applied to also disrupt and eradicate bio-film within the wound environment

3. The therapy of claim 1 wherein the hydrophobic fabric is comprised of hydrophilic fibers treated with a hydrophobic coating.

4. A wound treatment therapy comprising; a) applying hydrophobic wound dressing to a wound environment; b) saturating the hydrophobic wound dressing with a hypochlorous acid compound to deactivate micoorganisms without lysing pathogens; and c) removing the hydrophobic wound dressing from the wound environment to remove the deactivated microorganisms.

5. The therapy of claim 4 wherein the hyprochlorous acid is applied to also disrupt and eradicate bio-film within the wound environment

6. The therapy of claim 4 wherein the hydrophobic fabric is comprised of hydrophilic fibers treated with a hydrophobic coating.
